(19) United States
(12) Patent Application Publication
(10) Pub. No.: US 2022/0233487 A1
(43) Pub. Date: Jul. 28, 2022

(54) PREPARATION FOR USE IN WEIGHT MANAGEMENT COMPRISING OMEGA-3 FATTY ACID SALTS AND BASIC AMINO ACIDS

(71) Applicant: Evonik Operations GmbH, Essen (DE)

(72) Inventors: Rentje Frederik WITKAMP, LM Wijk bij Duurstede (NL); Michiel Gerard Juliaan BALVERS, SN Wageningen (NL)

(73) Assignee: Evonik Operations GmbH, Essen (DE)

(21) Appl. No.: 17/608,524

(22) PCT Filed: Apr. 30, 2020
(86) PCT No.: PCT/EP2020/061976

§ 371 Date: Nov. 3, 2021

(30) Foreign Application Priority Data
May 6, 2019 (EP) 19172780.9

(51) Int. Cl. A61K 31/202; A61K 31/201; A61K 31/198

(52) U.S. Cl. CPC A61K 31/202; A61K 31/198; A61K 31/201

(57) ABSTRACT

The present invention is directed to preparation comprising at least one a salt made up from fatty acids containing at least one omega-3 fatty acid and basic amino acids for use in stimulating an increased secretion of satiety hormones in a subject.

This invention concerns a method for stimulating secretion of certain satiety hormones in mammals upon administration of a product containing a salt made up from fatty acids containing omega-3 fatty acids and basic amino acids, resulting in the secretion of certain satiety hormones in the body of said mammal, leading to an increased feeling of satiety. As a consequence, less food is consumed which finally results in reduced weight gain or even actual weight loss.

Overweight and obesity have developed into serious societal problems not only in the western world, but all over the globe. A variety of health concerns and diseases are directly or indirectly related to them, including cardiovascular diseases (CVD), metabolic diseases such as type-2 diabetes, impairments of the skeletal system such as joint problems, and many more. These do not only negatively impact on the quality of life of affected people, but also put an immense burden on public and private health care systems. As a consequence, there is a growing demand for novel strategies to address these issues, beyond simple and in many cases fruitless dietary suggestions.

Various hormones have been described to regulate the intake of food. Ghrelin is a 28-mer peptide which has an orexigenic effect. In contrast, some other hormones, such as cholecystokinin (CCK), glucagon-like peptide-1 (GLP-1), the 36-mer peptide YY (PYY), nesfatin-1, adiponectin or leptin rather create a feeling of satiety. Therefore, stimulation of these satiety hormones is a possible way to reduce food intake and, as a consequence, manage weight gain and counteract civilization diseases such as obesity, diabetes, hypertension etc.

n-3 Long chain poly-unsaturated fatty acids (n-3 LC PUFAs) play vital roles as components of biological membranes and precursors of many important signalling molecules. As humans do not have enzymes to insert a double bond in the n-3 position, n-3 (and also n-6) fatty acids are essential dietary components [Wdtkamp, R. F., The role of fatty acids and their endocannabinoid-like derivatives in the molecular regulation of appetite. Mol Aspects Med, 2018. 64: p. 45-67]. Main n-3 LC PUFAs in the human diet are α-linolenic acid (ALA; 18:3n-3), predominantly obtained from plant sources, and the ‘marine’ forms eicosapentaenoic acid (EPA; 20:5n-3), docosahexaenoic acid (DHA; 22:6n-3) and, to a lesser extent, docosapentaenoic acid (DPA; 22:5n-3), which are particularly found in “fatty” fish (e.g. herring, salmon, mackerel) as well as in certain algae and krill [Calder, P. C., Very long-chain n-3 fatty acids and human health: fact, fiction and the future. Proceedings of the Nutrition Society, 2017: p. 1-21]. Endogenous conversion of ALA to EPA and DHA is very limited in humans, in particular in adults [Brenna, J. T., et al., α-Linolenic acid supplementation and conversion to n-3 long-chain polyunsaturated fatty acids in humans. Prostaglandins, Leukotrienes and Essential Fatty Acids, 2009. 80(2-3): p. 85-91]. Intake of n-3 LC PUFAs, in particular DHA and (or) EPA has been associated with a variety of positive health effects. Examples include improvement of endothelial function, lowered plasma triglyceride levels, a reduced risk for ischemic stroke, neuroprotective and antidepressant effects, prevention of cognitive decline, positive effects in rheumatoid arthritis, fatty liver disease, cancer-associated cachexia etc.

Several explanations have been brought forward for these apparent discrepancies. For example, it has been suggested that dose, time of intake and presence of other fatty acids in the diet, like n-6 PUFAs, differences between EPA and DHA, and interindividual differences-such as polymorphisms, sex, and age-are also playing a role [Zhuang, P., et al., Polyunsaturated fatty acids intake, omega-Momega-3 rato and mortality: Findings from two independent nationwide cohorts. Clinical Nutrition, 2018]. Because of these positive health effects, authorities recommend to consume at least 200 mg of DHA+EPA per day, which would require at least approximately 1 portion of fatty fish per week. However, in many populations and individuals, intakes of EPA and DHA from dietary sources are well below recommendations. Furthermore, several lines of evidence suggest that higher amounts are sometimes preferred, in particular for specific clinical purposes [Calder, P. C., Fatty acids and inflammation: The cutting edge between food and pharma. European Journal of Pharmacology, 2011. 668, Supplement 1(0): p. S50-S58]. Together, this generates an increasing demand to supply these n-3 LC PUFAs as food supplements, via pharmaceutical preparations or in other products.

EPA and DHA, either from the diet or as (supplement) preparation, are usually ingested as triglyceride (tri-acylglycerols (TAG), natural or re-esterified (rTAG)), or phospholipids (and sometimes mixtures thereof). Supplements or medicinal products may also contain ethyl-esters or free fatty acids. Once ingested, the esterified forms of fatty acids require enzymatic hydrolysis by lipases released by the pancreas into the duodenum, resulting in the formation of mono-acylglycerols and free fatty acids.

Salts of omega-3 fatty acids and amino acids have been known for quite some time already (S. El Boustani, C. Colette, L. Monnier, B. Descomps, A. Crastes de Paulet, F. Mendy, Lipids 1987, 22, 711-714; L. Monnier, S. El Boustani, A. Crastes de Paulet, B. Descomps, F. Mendy, Revue Francaise des Corps Gras 1989, 36, 3-10; T. Bruzzese, E P 0734373 B1; B. L. Mylari, F. C. Sciavolino, WO 2014011895A2). Recently, lysine salt complexes of EPA and DHA have been developed as powders with favourable stability, sensory and technical properties, which have been described to be particularly stable towards oxidation (G. Knaup, M. Latinovic, M. Schwarm, W O 2016/102323 A1).

Following their ingestion, these complexes dissociate in the acidic environment of the stomach, allowing the free fatty acids to be absorbed from the small intestinal tract. Independent of their original formulation, the free fatty acids will be incorporated into micelles in the intestinal lumen. Subsequently, EPA and DHA are absorbed via simple diffusion or via transport mechanisms involving CD38/FABP or FATP4 [Buttet, M., et al., From fatty-acid sensing to chylomicron synthesis: Role of intestinal lipid-binding proteins. Biochimie, 2014. 96: p. 37-47; Wang, T. Y., et al., New insights into the molecular mechanism of intestinal fatty acid absorption. European journal of clinical investigation, 2013.43(11): p. 1203-1223]. Inside the enterocyte, fatty acids are used to 35 synthesize triglycerides. Next, these triglycerides are packaged with cholesterol, lipoproteins and other lipids into chylomicrons. Chylomicrons are transported first through the lymphatic system and delivered into the blood circulation via the thoracic duct. In the circulation, fatty acids can be bound to/incorporated in different pools: as free fatty acids (non-covalently bound to albumin), TAGs and cholesteryl-esters in circulating triglyceride-rich lipoproteins, chylomicrons, very low-density lipoproteins, erythrocytes etc. [Browning, L. M., et al., Incorporation of eicosapentaenoic and docosahexaenoic acids into lipid pools when given as supplements providing doses equivalent to typical intakes of oily fish. The American Journal of Clinical Nutrition, 2012. 96(4): p. 748-758; Risé, P., et al., Fatty acid composition of plasma, blood cells and whole blood: Relevance for the assessment of the fatty acid status in humans. Prostaglandins Leukotrienes and Essential Fatty Acids, 2007. 76(6): p. 363-369].

It is well known that free fatty acids (FFAs) stimulate the release of satiety hormones such as cholecystokinin (CCK) and GLP-1 from the gastrointestinal tract (T. Vilsboll et al., J. Clin. Endocrinol. Metab. 2003, 88, 2706-2713; J. J. Holst, C. Orskov, Scand. J. Clin. Lab. Invest. Suppl. 2001, 234, 75-85; R. Guimbaud et al., Pancreas 1997, 14, 76-82). Recently, it was found that unsaturated long-chain FFAs had a stimulating effect on GLP-1 secretion in STC-1 from mice, which was confirmed in vivo (A. Hirasawa, K. Tsumaya, T. Awaji, S. Katsuma, T. Adachi, M. Yamada, Y. Sugimoto, S. Miyazaki, G. Tsujimoto, Nature Medicine 2005, 11, 90-94). Longer and polyunsaturated fatty acids (LC-PUFAs) such as EPA were described to lead to a decrease in preproghrelin and an increase in nucb2/nesfatin-1 expression in in goldfish hepatocytes (J. I. Bertucci, A. M. Blanco, L. F. Canosa, S. Unniappan, Comparative Biochemistry and Physiology, Part A: Molecular and Integrative Physiology 2017, 206, 24-35). LC-PUFAs such as EPA and DHA were also found to stimulate adiponectin secretion in human primary adipocytes (T. Romacho, P. Glosse, I. Richter, M. Elsen, M. H. Schoemaker, E. A. van Tol, J. Eckel, Nutrients 2015, 7, 865-886).

WO 2016/187643 relates to compositions comprising Pinus pinaster stem bark extract, papain and Aloe vera extract and to its use in improving health, for example, regulating blood sugar levels and treating, delaying or preventing conditions associated with or caused by elevated blood sugar levels. The claimed compositions may comprise further components selected from omega-3 fatty acids, phytonutrients, protein sources, amino acids, antioxidants, vitamins, minerals, plant extracts, and mixtures thereof. US 2011/0046053 describes oral pharmaceutical compositions comprising exenatide, a glucagon-like peptide (GLP-1) agonist, which is an incretin mimetic and potentiates exenatide secretion while inhibiting glucagon secretion and slowing gastric emptying. The composition further comprises a protease inhibitor, and an omega-3 fatty acid for treating diabetes mellitus.

JP2019026585A discloses a method for suppressing the production and secretion of active ghrelin by using docosahexaenoic acid (DHA). In cell studies it was shown that DHA influenced octanoyl ghrelin production and secretion, which was confirmed in mice experiments with DHA. However, in these studies LC-PUFAs were employed as free fatty acids (FFA), which have some difficulties in practical application. Free LC-PUFAs are particularly sensitive to oxidation, thus handling under oxygen-free conditions is mandatory to prevent rapid degradation. In addition, these free LC-PUFAs are oily liquids which are difficult to formulate for administration to humans as food supplements or pharmaceutical drugs.

Therefore, it was desirable to have a preparation with the ability to stimulate the release of satiety hormones in the body, while at the same time avoiding the disadvantages of handling liquid oils which are difficult to formulate and highly sensitive to oxidative degradation.

The present invention is thus directed to preparation comprising at least one salt made up from fatty acids containing at least one omega-3 fatty acid and basic amino acids for use in stimulating an increased secretion of satiety hormones in a subject.

In this invention it was found that administration of such stable omega-3 fatty acid amino acid salts to human beings results in increased secretion of satiety hormones CCK, GLP-1 and PYY, as compared to the administration of a standard omega-3 ethyl ester preparation. Interestingly, no significant differences were seen for ghrelin plasma levels.

A further aspect of the present invention is directed to a preparation comprising at least one salt made up from fatty acids containing at least one omega-3 fatty acid and basic amino acids for treating or preventing a disease or disorder selected from obesity, adipositas, type 2 diabetes, metabolic syndrome.

In an advantageous configuration, the secreted satiety hormones are either CCK, GLP-1 and/or PYY.

According to the present invention, the subject may be a mammal, preferably selected from humans, dogs or cats.

The preparation according to the present invention is preferably a functional/fortified food, a dietary supplement or a pharmaceutical drug.

In a preferred configuration the salt is made up from fatty acids comprising omega-3 fatty acids and basic amino acids in an almost equimolar ratio ranging from 0.9:1.1 to 1.1-0.9.

In a further preferred configuration of the present invention, the omega-3 fatty acids are selected from alpha-linoleic acid (ALA), stearidonic acid (SDA), eicosapentaenoic acid (EPA), docosapentaenoic acid (DPA) or docosahexaenoic acid (DHA), preferably selected from EPA and DHA. It is preferred, when the omega-3 fatty acids comprise EPA and DHA, preferably in a molar ratio EPA/DHA of between 0.50 and 3.00, more preferably in a ratio EPA/DHA of between 1.00 and 2.00.

The basic amino acid may be selected from lysine, arginine or omithine, preferably lysine and it is preferred when the basic amino acids are selected from L-lysine, L-arginine or L-omithine, preferably L-lysine.

In fact, the basic amino acids lysine, arginine or omithine have similar characteristics, all being basic, charged (at physiological pH), aliphatic amino acids, with similar pKₐ-values:

| Basic amino acid | L-lysine | L-arginine | L-ornithine |
|---|---|---|---|
| Chemical formula | C₆H₁₄N₂O₂ | C₆H₁₄N₄O₂ | C₅H₁₂N₂O₂ |
| Molar mass | 146.19 g/mol | 174.20 g/mol | 132.2 g/mol |
| pKₐ-values | pK_{COOH}: 2.20 | pK_{COOH}: 2.0 | pK_{COOH}: 1.94 |
| | pK_{alpha-NH2}: 8.9 | pK_{alpha-NH2}: 9.0 | pK_{alpha-NH2}: 8.65 |
| | pK_{epsilon-group}: 10.28 | pK_{Guanidin-group}: 12.1 | pK_{delta-NH2}: 10.76 |

Another aspect of the present invention is related to a preparation comprising at least one salt made up from fatty acids containing at least one omega-3 fatty acid and basic amino acids for use in resulting in a feeling of satiety in the respective subject.

Another aspect of the present invention is related to a preparation comprising at least one salt made up from fatty acids containing at least one omega-3 fatty acid and basic amino acids for use in resulting in reduced food intake of the respective subject.

Another aspect of the present invention is related to a preparation comprising at least one salt made up from fatty acids containing at least one omega-3 fatty acid and basic amino acids for use in resulting reduced weight gain or weight loss in the respective mammal.

### WORKING EXAMPLES

Ethical Approval

This study was approved by the medical ethical committee of Wageningen University (METC-WU; NL 63619.081.18) and conducted in accordance with the principles of the Declaration of Helsinki (64th WMA General Assembly, Fortaleza, Brazil, October 2013) and with the Medical Research Involving Human Subjects Act (WMO). All subjects gave their written informed consent.

Subjects

Eight healthy women volunteered to participate in this study and they provided a full-written informed consent. Their mean (±S.D.) age was 23.4±1.5 years, height 1.69±7 cm, weight 62.0 t 5.8 kg and BMI 21.7±2.0 kg/m2. They had been included based on BMI (18.5−25 kg/m2), age, general health and being able to donate blood samples. Relevant exclusion criteria were current diseases, any gastrointestinal conditions/diseases within the 3 months prior to the intervention, use of medication two months before and during the intervention, except for oral contraceptives and occasional use of painkillers, reported weight loss or weight gain of >2 kg in the month prior to the intervention, use of omega-3 or fish oil supplements, 3 weeks before-, or during the intervention, allergies to test products (shell)fish or soy products), drug abuse, smoking, alcohol consumption of >10 glasses per week, recent or planned blood donation (<4 month prior to first study day or during intervention), haemoglobin (Hb) level<7.5 mmol/L, been pregnant or breastfeeding in the last 6 months, or plan to become pregnant or breastfeed during the study and (planned or recent) participation in other research.

Study Design

The study had a cross-over design with each participant acting as her own control. The 2 experimental sessions lasted 48h and were separated by a wash-out period of one week. To reduce the effect of disparities between groups due to potential drop-out in advance, both preparations were tested in 4 subjects during each session. Participants were randomly allocated to a test sequence. During a session, they were provided standardized meals at T=2, 6, 11, 24, 28 and 34 hours after ingestion of the study products. A standardized snack was provided at T=9.

After the sampling period, the second morning (48h), subjects were provided with a breakfast.

On each first test day, participants arrived at the research facility in the morning in a fasted state. The evening before they were requested to consume a standardized meal. After placement of a venous catheter and donating a baseline blood sample (T=0), subjects received either the EPA and DHA lysine preparation, or EPA and DHA as a fish oil food supplement, both in the form of capsules, with 150 ml of tap water. Subsequently, blood samples (9 ml until T=4h, 6 ml at the remaining timepoints) were taken at T=0.5, 1, 2, 3, 4, 5, 6, 8, 10, 12, 14, 16, 20, 24, 32, 48 hours after ingestion.

### Study Products and Dosing

Research subjects received either a preparation of lysine salts of EPA and DHA (AvailOm®, Evonik Nutrition and Care GmbH, Germany), or a conventional fish oil food supplement containing EPA and DHA (LUCOVITAAL, Puur Koudwater Omega 3 Visolie, Holland and Barrett, Ede, The Netherlands) on each occasion. The lysine salt preparation was administered in a dose of 1400 mg, given in 7 capsules (size no 2) containing 200 mg each. The fish oil supplement was given as a single oral dose of 1400 mg, in a softgel capsule as supplied. The lysine salt preparation contained per 1400 mg dose: 499.8 mg EPA and 302.4 mg DHA (EPA/DHA molar ration 1.79). The fish oil supplement specifications were per dose of 1400 mg: 504 mg EPA and 378 mg DHA (EPA/DHA molar ration 1.44). Before administration, the fish oil supplement was analyzed to confirm fatty acid composition. Furthermore, to establish the molecular form, NMR was performed. It was found that it consisted largely of ethylesters.

Plasma Fatty Acid Analysis

Blood samples (6 or 9 ml) were collected in blood collection tubes. Plasma (EDTA-aprotinin) was separated and stored at −80° C. until further analysis. A sub-sample of the intervention products is also stored and included in the analysis. Free and total esterified (cholesterol, phospholipids and glycerol-esters) DHA and EPA levels in plasma were analyzed using gas chromatography (GC) using 650 μL plasma per sample. Briefly, fat fractions were extracted using hexane, and purified by solid phase extraction using silica columns. Next, fatty acids were derivatized to methylesters by the boron trifluoride-methanol transesterification method. Methylesters were separated by capillary GC and detected by flame-ionisation. Concentrations were calculated via single-point calibration, using a C19-TAG as standard. Per batch of 48 samples, 3 QC samples (one blanc, 2 spiked) were co-analyzed in duplicate. These were prepared from a batch of quality control plasma present in the lab. The intra-batch coefficient of variability (CV) for DHA-TAG was 13%, for EPA 17%.

Analysis of Satiety Hormones

The satiety hormones, Ghrelin, GLP-1, PYY, were analyzed in EDTA plasma prepared from blood to which 50 μL DPP IV inhibitor (DPP4-010 Sigma-Aldrich) per ml of blood had been added. The hormones were analyzed using ELISA kits from Millipore (human total PYY cat no #EZHPYYT66K; human GLP-1 cat no #EZGLPIT-36K; human Ghrelin cat no #EZGRT-89K) according to the manufacturer's instruction.

Data Handling and Data Analysis

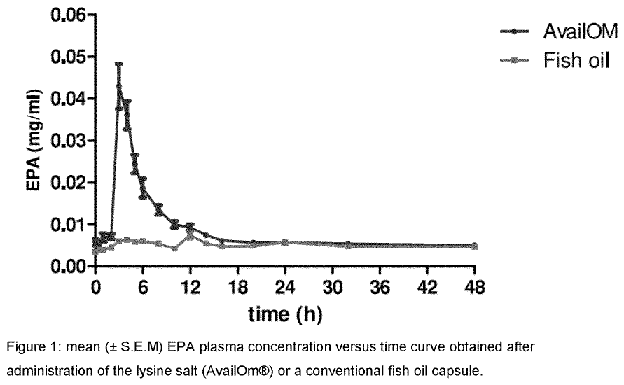

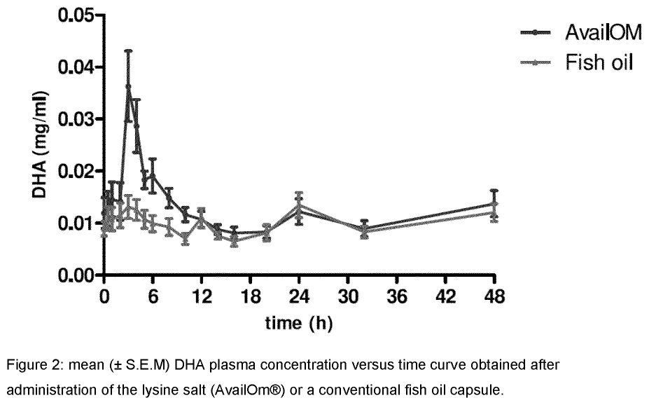

Maximum peak height (Cmax), time-to-peak (Tmax) were directly estimated from the individual plasma curves. Areas-under-the-curve (AUC) from T=0 until T=20 h were calculated for each separately by the trapezoid rule using GraphPad Prism version 5.00 for Windows, GraphPad Software, San Diego Calif. USA. This time interval was selected from the observation that curves obtained after the different products were superimposable (FIGS. 1 and 2) after that time point.

### Example 1: Determination of Plasma Concentrations of DHA and EPA

In a human study the short-term oral bioavailability and plasma kinetics of EPA and DHA following single oral administration, after an overnight fast, of a mixture of the lysine salts of EPA (500 mg fatty acid) and of DHA (300 mg fatty acid), in comparison to commercially available fish oil to 8 healthy young women was investigated.

The total (free and esterified) DHA and EPA plasma concentrations was determined for all 8 participants after ingestion of either a preparation of lysine salts of EPA and DHA or a fish oil food supplement containing EPA and DHA. Individual fatty acid plasma concentration versus time curves were plotted for each participant (supplemental FIGS. 1 and 2). Although some variation was seen in absolute plasma levels, in particular with DHA, their time-course showed a high degree of similarity. After administration of the lysine salts, rather sharp peak plasma concentrations were reached after approximately 3 hours, for both EPA and DHA. In 2 participants (5 and 8) the peak came slightly later, approximately at 4 and 5 hours following administration of the lysine salts. After administration of the conventional fish oil preparation, plasma values showed a small increment, but no clear peaks were seen. For both preparations, a second small peak was sometimes seen at approximately 24 h following administration. FIG. 1 shows the mean (±S.E.M) EPA plasma concentration versus time curve obtained after administration of the lysine salt (AvailOm®) or a conventional fish oil capsule. FIG. 2 shows mean (±S.E.M) DHA plasma concentration versus time curve obtained after administration of the lysine salt (AvailOm®) or a conventional fish oil capsule. From t=20 onwards, average plasma curves were found to overlap completely for both conditions. Areas-under-the-curve (AUC) were determined both for EPA and DHA. The results for the 8 participants are shown in tables 1 and 2.

TABLE 1
AUC values (+SD) for EPA from 0-20 h

| | |
|---|---|
| EPA | AUC (mg/ml · h) |

| Participant | Lysine salt | Fish oil | ratio |
|---|---|---|---|
| 1 | 0.1645 | 0.04638 | 3.546787408 |
| 2 | 0.1283 | 0.01585 | 8.094637224 |
| 3 | 0.204 | 0.06455 | 3.160340821 |
| 4 | 0.2118 | 0.01406 | 15.06401138 |
| 5 | 0.1627 | 0.04702 | 3.460229689 |
| 6 | 0.07496 | 0.03249 | 2.307171437 |
| 7 | 0.2339 | 0.05911 | 3.957029267 |
| 8 | 0.1203 | 0.05352 | 2.247757848 |
| mean | 0.1625575 | 0.0416225 | 3.905519851 |
| | | dose | 3.93676401 |
| | | correction | |

TABLE 2
AUC values (+SD) for DHA from 0-20 h

| | |
|---|---|
| DHA | AUC (mg/ml · h) |

| Participant | Lysine salt | Fish oil | ratio |
|---|---|---|---|
| 1 | 0.1384 | 0.07312 | 1.892778993 |
| 2 | 0.07823 | 0.0422 | 1.853791469 |
| 3 | 0.1081 | 0.0303 | 3.567656766 |
| 4 | 0.1309 | 0.04013 | 3.261898829 |
| 5 | 0.08615 | 0.0378 | 2.279100529 |
| 6 | 0.07803 | 0.0602 | 1.296179402 |
| 7 | 0.367 | 0.1237 | 2.966855295 |
| 8 | 0.06412 | 0.01456 | 4.403846154 |
| mean | 0.13136625 | 0.05275125 | 2.490296438 |
| | | dose | 3.116993555 |
| | | correction | |

For EPA, mean AUC values (±SD) from 0-20 h were 0.163±0.053 mg.l-1.h after the lysine salt, and 0.042±0.019 mg.l-1.h after the fish oil preparation, which corresponds to an estimated relative oral bioavailability of 3.9 for EPA when administered as the lysine salt compared to the fish oil (see table 1). For DHA, mean estimated AUC values (±SD) from 0-20 h were 0.1314±0.099 mg.l-1.h and 0.054±0.034 mg.l-1.h for the lysine salt and the fish oil preparation, respectively. This corresponds to a relative oral bioavailability of 3.1 (including dose correction) of the lysine salt compared to the fish oil. (see table 2).

It could be shown that both fatty acids are rapidly and well absorbed. Although the present design does not allow determination of the absolute bioavailability (i.e. relative to that after i.v administration), the relative bio-availability compared to that of the comparator preparation was on average 3.9 and 3.1 times higher for EPA and DHA, respectively.

### Example 2: Determination of Plasma Concentrations of Satiety Hormones

In a next step it was investigated, whether the availability of free EPA and DHA in the upper duodenum would affect plasma profiles of satiety hormones (Ghrelin, GLP-1 and PYY) resulting from interaction with Free Fatty Acid Receptors (FFA1-FFA4) and other receptors that can be activated by these fatty acids. Direct chemosensing of fatty acids takes place via interaction with free fatty acid (FFA) and other receptors which are present along the entire gastrointestinal tract [Witkamp, R. F., The role of fatty acids and their endocannabinoid-like derivatives in the molecular regulation of appetite. Mol Aspects Med, 2018. 64: p. 45-67]. Stimulation of these receptors leads to several neural, paracrine and endocrine responses, including induction of satiation (during a meal) and satiety (between meals). This might have interesting consequences for food-intake.

Therefore, plasma-time profiles of the satiety hormones Ghrelin, GLP-1 and PYY were determined after ingestion of either a preparation of lysine salts of EPA and DHA or a fish oil food supplement containing EPA and DHA.

TABLE 3
Ghrelin plasma values (pg/ml) after administration
of lysine salt or fish oil

| Participant | Time (h) | Lysine salt | | Fish oil | |
|---|---|---|---|---|---|
| 1 | 0 | 593.45 | | 791.23 | |
| | 0.5 | 695.71 | | 855.34 | |
| | 1 | 741.60 | | 929.69 | |
| | 2 | 744.30 | | 941.08 | |
| | 3 | 537.43 | | 591.40 | |
| | 4 | 436.37 | | 486.21 | |
| 2 | 0 | 834.03 | | 749.56 | |
| | 0.5 | 946.02 | | 761.40 | |
| | 1 | 1018.99 | | 777.18 | |
| | 2 | 1035.50 | | 741.96 | |
| | 3 | 518.62 | | 475.77 | |
| | 4 | 444.49 | | 403.91 | |
| 3 | 0 | 547.22 | | 481.94 | |
| | 0.5 | 541.55 | | 516.09 | |
| | 1 | 579.68 | | 556.47 | |
| | 2 | 540.41 | | 574.79 | |
| | 3 | 379.16 | | 409.03 | |
| | 4 | 337.10 | | 336.45 | |
| 4 | 0 | 1105.79 | | 1130.57 | |
| | 0.5 | 1243.48 | | 1260.72 | |
| | 1 | 1025.48 | | 1319.69 | |
| | 2 | 1153.59 | | 1287.45 | |
| | 3 | 735.12 | | 899.38 | |
| | 4 | 669.13 | | 697.95 | |
| 5 | 0 | 215.65 | | 199.15 | |
| | 0.5 | 258.97 | | 224.91 | |
| | 1 | 263.81 | | 231.26 | |
| | 2 | 254.46 | | 241.42 | |
| | 3 | 231.41 | | 194.28 | |
| | 4 | 196.52 | | 180.90 | |
| 6 | 0 | 999.51 | | 887.88 | |
| | 0.5 | 1044.22 | | 907.51 | |
| | 1 | 988.00 | | 890.32 | |
| | 2 | 932.94 | | 761.35 | |
| | 3 | 587.40 | | 659.51 | |
| | 4 | 439.50 | | 510.29 | |
| 7 | 0 | 254.11 | | 220.12 | |
| | 0.5 | 221.11 | | 225.67 | |
| | 1 | 226.84 | | 229.12 | |
| | 2 | 222.76 | | 225.46 | |
| | 3 | 207.40 | | 219.18 | |
| | 4 | 153.16 | | 184.53 | |
| 8 | 0 | 653.20 | | 670.07 | |
| | 0.5 | 577.69 | | 655.98 | |
| | 1 | 642.18 | | 648.64 | |
| | 2 | 657.35 | | 605.87 | |
| | 3 | 427.29 | | 472.73 | |
| | 4 | 356.23 | | 416.89 | |
| Means ± | 0 | 650.37 | ±113.62 | 641.32 | ±114.45 |
| S.E.M | 0.5 | 691.09 | ±129.40 | 675.95 | ±124.44 |
| | 1 | 685.82 | ±113.60 | 697.80 | ±129.81 |
| | 2 | 692.67 | ±121.57 | 672.42 | ±124.12 |
| | 3 | 452.98 | ±63.33 | 490.16 | ±81.77 |
| | 4 | 379.06 | ±57.01 | 402.14 | ±60.81 |

TABLE 4
GLP-1 plasma values (pg/ml) after administration
of lysine salt or fish oil

| Participant | Time (h) | Lysine salt | Fish oil |
|---|---|---|---|
| 1 | 0 | 27.00 | 17.70 |
| | 0.5 | 23.66 | 18.80 |
| | 1 | 44.58 | 21.38 |
| | 2 | 30.72 | 18.77 |
| | 3 | 42.43 | 44.98 |
| | 4 | 43.57 | 35.81 |
| 2 | 0 | 17.80 | 23.40 |
| | 0.5 | 15.85 | 23.90 |
| | 1 | 19.59 | 20.25 |
| | 2 | 20.59 | 21.44 |
| | 3 | 59.24 | 51.25 |
| | 4 | 59.69 | 45.58 |
| 3 | 0 | 17.66 | 20.95 |
| | 0.5 | 15.99 | 15.60 |
| | 1 | 18.32 | 17.17 |
| | 2 | 17.23 | 16.32 |
| | 3 | 58.24 | 22.48 |
| | 4 | 27.24 | 19.47 |
| 4 | 0 | 32.59 | 28.23 |
| | 0.5 | 32.74 | 26.49 |
| | 1 | 39.71 | 22.05 |
| | 2 | 34.97 | 22.22 |
| | 3 | 58.27 | 29.93 |
| | 4 | 39.32 | 39.45 |
| 5 | 0 | 25.45 | 21.96 |
| | 0.5 | 25.56 | 23.88 |
| | 1 | 38.13 | 24.94 |
| | 2 | 31.92 | 17.37 |
| | 3 | 38.73 | 36.53 |
| | 4 | 42.19 | 35.79 |
| 6 | 0 | 12.74 | 17.18 |
| | 0.5 | 14.07 | 14.73 |
| | 1 | 22.15 | 15.02 |
| | 2 | 15.16 | 12.30 |
| | 3 | 37.53 | 16.81 |
| | 4 | 42.32 | 27.32 |
| 7 | 0 | 10.08 | 12.03 |
| | 0.5 | 15.89 | 9.24 |
| | 1 | 8.30 | 20.49 |
| | 2 | 7.12 | 18.31 |
| | 3 | 30.13 | 31.90 |
| | 4 | 25.45 | 35.45 |
| 8 | 0 | 16.62 | 15.66 |
| | 0.5 | 14.95 | 19.46 |
| | 1 | 12.28 | 19.17 |
| | 2 | 12.28 | 19.87 |
| | 3 | 23.82 | 37.21 |
| | 4 | 25.72 | 37.91 |

| | | | | | |
|---|---|---|---|---|---|
| Means ± | 0 | 19.99 | ±2.53 | 19.64 | ±1.79 |
| S.E.M | 0.5 | 19.84 | ±2.23 | 19.01 | ±2.02 |
| | 1 | 25.38 | ±4.50 | 20.06 | ±1.07 |
| | 2 | 21.25 | ±3.37 | 18.32 | ±1.11 |
| | 3 | 43.55 | ±4.52 | 33.89 | ±3.97 |
| | 4 | 38.19 | ±3.88 | 34.60 | ±2.81 |

TABLE 5
PYY plasma values (pg/ml) after administration
of lysine salt or fish oil

| Participant | Time (h) | Lysine salt | | Fish oil | |
|---|---|---|---|---|---|
| 1 | 0 | 46.70 | | 28.05 | |
| | 0.5 | 34.38 | | 24.92 | |
| | 1 | 65.17 | | 18.49 | |
| | 2 | 76.21 | | 18.25 | |
| | 3 | 77.40 | | 47.74 | |
| | 4 | 108.57 | | 70.91 | |
| 2 | 0 | 41.04 | | 59.85 | |
| | 0.5 | 30.47 | | 62.25 | |
| | 1 | 43.65 | | 53.59 | |
| | 2 | 48.12 | | 56.24 | |
| | 3 | 117.40 | | 108.81 | |
| | 4 | 114.13 | | 113.36 | |
| 3 | 0 | 37.99 | | 43.17 | |
| | 0.5 | 34.58 | | 36.20 | |
| | 1 | 33.26 | | 36.10 | |
| | 2 | 27.73 | | 30.68 | |
| | 3 | 56.33 | | 42.01 | |
| | 4 | 43.94 | | 51.20 | |
| 4 | 0 | 83.77 | | 53.13 | |
| | 0.5 | 68.14 | | 57.96 | |
| | 1 | 78.58 | | 54.69 | |
| | 2 | 101.84 | | 45.66 | |
| | 3 | 141.11 | | 97.59 | |
| | 4 | 136.46 | | 91.35 | |
| 5 | 0 | 37.99 | | 69.70 | |
| | 0.5 | 45.85 | | 51.57 | |
| | 1 | 35.49 | | 54.14 | |
| | 2 | 29.84 | | 40.35 | |
| | 3 | 65.78 | | 60.65 | |
| | 4 | 75.36 | | 65.69 | |
| 6 | 0 | 51.10 | | 69.61 | |
| | 0.5 | 39.47 | | 85.02 | |
| | 1 | 41.91 | | 66.92 | |
| | 2 | 31.61 | | 60.02 | |
| | 3 | 132.17 | | 82.02 | |
| | 4 | 109.20 | | 95.82 | |
| 7 | 0 | 36.80 | | 64.90 | |
| | 0.5 | 33.16 | | 56.97 | |
| | 1 | 49.71 | | 52.03 | |
| | 2 | 73.74 | | 41.04 | |
| | 3 | 118.64 | | 80.18 | |
| | 4 | 116.78 | | 95.82 | |
| 8 | 0 | 30.05 | | 39.67 | |
| | 0.5 | 40.45 | | 35.49 | |
| | 1 | 57.15 | | 33.57 | |
| | 2 | 48.68 | | 35.69 | |
| | 3 | 123.87 | | 50.27 | |
| | 4 | 155.31 | | 84.27 | |
| Means ± | 0 | 45.68 | ±5.89 | 53.51 | ±5.41 |
| S.E.M | 0.5 | 40.81 | ±4.27 | 51.30 | ±6.69 |
| | 1 | 50.61 | ±5.50 | 46.19 | ±5.48 |
| | 2 | 54.72 | ±9.45 | 40.99 | ±4.76 |
| | 3 | 104.09 | ±11.50 | 71.16 | ±8.72 |
| | 4 | 107.47 | ±12.20 | 83.55 | ±7.04 |

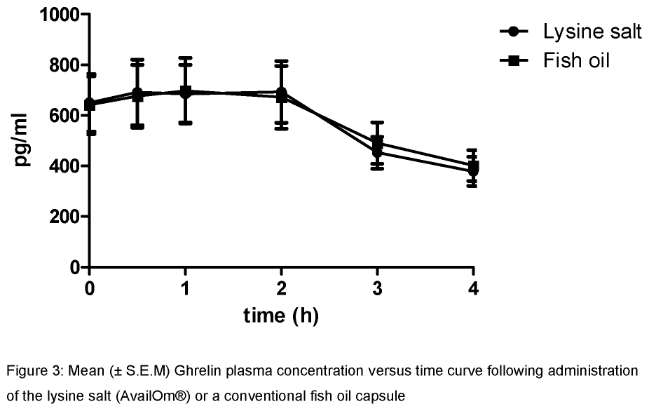

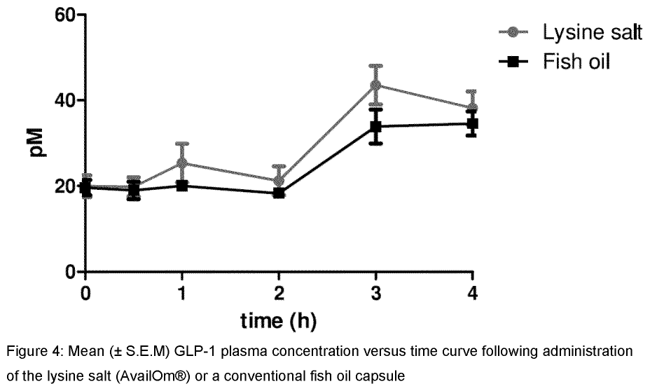

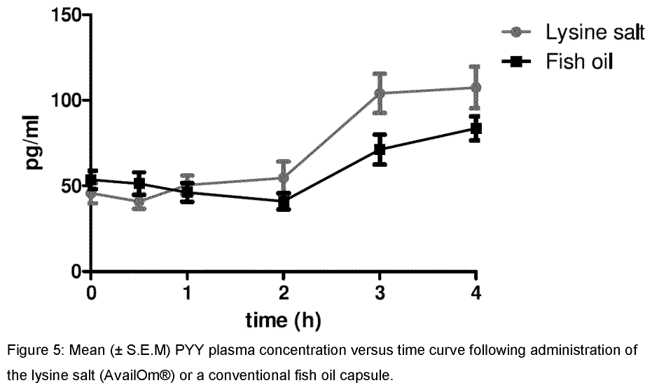

FIG. 3 shows the mean (±S.E.M) Ghrelin plasma concentration versus time curve following administration of the lysine salt (AvailOm®) or a conventional fish oil capsule. FIG. 4 shows the mean (±S.E.M) GLP-1 plasma concentration versus time curve following administration of the lysine salt (AvailOM®) or a conventional fish oil capsule and FIG. 5 shows mean (±S.E.M) PYY plasma concentration versus time curve following administration of the lysine salt (AvailOm®) or a conventional fish oil capsule.

Higher PYY and GLP-1 levels were seen after taking the lysine salts. This difference was particularity clear after the breakfast was taken, 2 hours after administering the n-3 LC PUFAs. These findings might indicate that the presence of free fatty acids in the upper duodenum stimulates satiation. No significant differences were seen for Ghrelin plasma levels.

1. A method for increasing a secretion of at least one satiety hormone, the method comprising:
administrating to a subject in need thereof a preparation comprising a salt of at least one fatty acid comprising an omega-3 fatty acid and at least one basic amino acid.

2. The method of claim 1, wherein the subject in need thereof has a disease or disorder selected from the group consisting of obesity, adipositas, type 2 diabetes and metabolic syndrome.

3. The method of claim 1, wherein the at least one secreted satiety hormone is at least one selected from the group consisting of CCK, GLP-1 and PYY.

4. The method of claim 1, wherein the subject in need thereof is a mammal.

5. The method of claim 1, wherein the preparation is a functional/fortified food, a dietary supplement or a pharmaceutical drug.

6. The method of claim 1, wherein the at least one fatty acid and the at least one basic amino acid in the salt is in a ratio ranging from 0.9:1.1 to 1.1-0.9.

7. The method of claim 1, wherein the omega-3 fatty acid is at least one selected from the group consisting of alpha-linoleic acid (ALA), stearidonic acid (SDA), eicosapentaenoic acid (EPA), docosapentaenoic acid (DPA) and docosahexaenoic acid (DHA).

8. The method of claim 1, wherein the omega-3 fatty acid comprises IPA and DHA.

9. The method of claim 1, wherein the at least one basic amino acid is at least one selected from the group consisting of lysine, arginine and ornithine.

10. The method of claim 1, wherein the at least one basic amino acid is at least one selected from the group consisting of L-lysine, L-arginine and L-ornithine.
